(19) United States
(12) Patent Application Publication
(10) Pub. No.: US 2007/0134650 A1
(43) Pub. Date: Jun. 14, 2007

(54) SCLERAXIS

(75) Inventor: Michael P. Czubryt, Winnipeg (CA)

Correspondence Address: BERESKIN AND PARR, 40 KING STREET WEST, BOX 401, TORONTO, ON, M5H 3Y2, CA

(73) Assignee: University of Manitoba, Winnipeg, CA (US)

(21) Appl. No.: 11/608,481

(22) Filed: Dec. 8, 2006

Related U.S. Application Data
(60) Provisional application No. 60/748,150, filed on Dec. 8, 2005.

(51) Int. Cl. A61K 48/00; C12Q 1/00

(52) U.S. Cl. 435/4; 514/44

(57) ABSTRACT

Methods and compositions for modulating collagen synthesis are described.

### CROSS-REFERENCE TO RELATED APPLICATIONS

This application claims the benefit under 35 USC §119(e) from U.S. provisional patent application Ser. No. 60/748,150, filed Dec. 8, 2005, which is incorporated herein by reference in its entirety.

### FIELD OF THE INVENTION

The present invention relates to methods and compositions for modulating collagen formation by modulating scleraxis activity or expression.

### BACKGROUND OF THE INVENTION

The cardiac extracellular matrix (ECM) is a complex scaffold that provides integrity for the walls of the heart, which must withstand large fluctuations in pressure on a beat-to-beat basis. The composition of the matrix provides both the strength to withstand high pressures, and the elasticity to permit adaptive changes in cardiac chamber dimensions. A key matrix component is collagen, which is synthesized by fibroblasts in the healthy heart and which provides structural strength and rigidity. In response to stresses such as hypertension, infarct or diabetes however, excessive collagen production can occur. This “fibrosis” is marked by a conversion of cardiac fibroblasts into myofibroblasts, which produce large amounts of collagen, resulting in stiffening of cardiac muscle and impaired function.

The inventor described an inducible transgenic mouse model of acute cardiac failure¹. It was noted at the time that several collagen genes, including fibrillar type I collagens which are major components of myocardial extracellular matrix in normal and fibrotic tissue, were strongly upregulated in mice with failing hearts (M. P. Czubryt and E. N. Olson, unpublished observations). It was also noted that the basic helix-loop-helix (bHLH) transcription factor scleraxis was upregulated as well, and this increase in scleraxis expression preceded induction of the collagen genes.

Scleraxis plays a key role in the development of tendons and cardiac valves, where its expression is associated with collagen synthesis^{2,3}. Furthermore, overexpression of scleraxis in osteoblastic ROS17/2.8 cells resulted in an increase in collagen II expression⁴. Scleraxis may thus directly regulate collagen gene expression, but this possibility has not yet been examined. A possible role for scleraxis in regulating cardiac matrix collagen expression also has not been studied.

### SUMMARY OF THE INVENTION

The present inventor has shown that scleraxis is involved in collagen synthesis and regulates the expression of collagen genes involved in cardiac fibrosis.

Accordingly, the present invention provides a method of modulating collagen synthesis comprising administering an effective amount of an agent that modulates scleraxis to a cell or animal in need thereof.

In one aspect, the present invention provides a method of inhibiting collagen synthesis comprising administering an effective amount of scleraxis antagonist to a cell or animal in need thereof. Preferably, the method is used to prevent or inhibit cardiac fibrosis.

In another aspect, the present invention provides a method of stimulating collagen synthesis comprising administering an effective amount of scleraxis agonist to a cell or animal in need thereof.

In yet another aspect, the present invention includes screening methods for identifying substances which are capable of modulating collagen synthesis by modulating scleraxis. In particular, the methods may be used to identify substances which are capable of augmenting or attenuating the effects of scleraxis (i.e. agonists). Alternatively, the methods may be used to identify substances which are capable of inhibiting the effects of scleraxis (i.e. antagonists).

The invention also includes screening assays for identifying agonists or antagonists of scleraxis comprising the steps of:

(a) incubating a test compound with a cell expressing scleraxis; and

(b) determining the effect of the compound on scleraxis activity or expression and comparing with a control (i.e. in the absence of the test compound), wherein a change in scleraxis activity or expression as compared to the control indicates that the test compound may modulate collagen synthesis.

The present invention also includes the pharmaceutical compositions comprising any of the above molecules that modulate scleraxis for use in modulating collagen synthesis. The pharmaceutical compositions preferably comprise a scleraxis antagonist. The pharmaceutical compositions can further comprise a suitable diluent or carrier.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1. Semi-quantitative RT-PCR of scleraxis. Total RNA was extracted from isolated adult rat cardiac myofibroblasts (P2). Primers specific for scleraxis were used in the following numbers of amplification cycles: 20, 22, 25, 27, 30, 32, 35 (lanes 2 through 8, respectively). Lane 1: 1 kb DNA ladder. The scleraxis PCR product is approximately 100 base pairs.

FIG. 2. Expression of scleraxis in adult rat cardiac myofibroblasts. Total RNA was isolated from cells or tissue using Trizol (see text). RT-PCR was performed using a Superscript III kit (Invitrogen) and the scleraxis-specific primers listed in the text, or primers specific for GAPDH. From left to right, the lanes represent samples from rat cardiac myofibroblasts, cardiomyocytes, liver or from COS-7 cells, respectively. The upper panel shows results using scleraxis-specific primers (30 cycles); the lower panel shows results for GAPDH (22 cycles).

FIG. 3. TGF-β1 induces scleraxis expression in neonatal rat cardiac fibroblasts. Primary neonatal rat cardiac fibroblasts were treated with 10 ng/ml TGF-β1or infected with adenoviruses encoding LacZ (Ad-LacZ) or scleraxis (Ad-Scx, MOI of 75 or 100 as indicated) for 24, 48 or 72 hours. Total RNA was isolated using Trizol and RT-PCR performed as in FIG. 2 using primers specific for scleraxis or GAPDH. TGF-β1 induces a time-dependent increase in scleraxis expression compared to Ad-LacZ infected cells. Ad-Scx-infected cells serve as positive controls, and demonstrate high efficiency expression of scleraxis after infection of fibroblasts.

FIG. 4. Regulation of collagen Iα2 expression by scleraxis. COS-7 cells were transiently transfected with a collagen Iα2 promoter reporter vector alone, or with reporter plus expression vectors encoding scleraxis, E12, E47 or MyoD. Scleraxis significantly upregulated expression of the reporter. E12 and E47 caused a small further induction, but these increases were not statistically significant compared to scleraxis alone. MyoD was unable to induce expression of the reporter gene. Luciferase expression levels were normalized to co-transfected β-galactosidase. Mean±standard error. *p<0.05 versus reporter, one-way ANOVA and Student-Newman-Keuls post-hoc test. n≧3.

FIG. 5. Involvement of endogenous bHLH factors in regulation of collagen Iα2 expression by scleraxis. COS-7 cells were transiently transfected with a collagen Iα2 promoter reporter vector alone, or with expression vectors encoding scleraxis or Id2. Upregulation of reporter gene expression was attenuated by cotransfection of the bHLH repressor Id2. Numbers in brackets are relative mass ratios of Id2 vector to scleraxis vector transfected. Luciferase expression levels were normalized to co-transfected β-galactosidase. Mean±standard error. *p<0.05 versus reporter alone. #p<0.05 versus scleraxis alone. n≧3. Statistical analysis as in FIG. 3.

FIG. 6. Increased expression of scleraxis in myofibroblasts compared to primary fibroblasts. Total RNA was extracted from isolated primary neonatal rat fibroblasts (P0) or myofibroblasts (P2). RT-PCR was performed using primers specific to scleraxis and 29 cycles of amplification. Results were normalized to GAPDH expression, and are representative of two individual experiments. Expression of scleraxis doubles as fibroblasts become myofibroblasts. Mean±standard error. *p<0.01vs. P0 by Student t-test. n≧3.

FIG. 7. Infection of rat cardiac myofibroblasts with AdLacZ. P2 adult rat cardiac myofibroblasts were untreated (control), or treated with an adenovirus encoding β-galactosidase (AdLacZ) at a multiplicity of infection of 10, 50 or 100. Twenty-four hours after infection, cells were stained using X-gal to indicate β-galactosidase activity. Using an MOI of 50, infected cells represent approximately 95% of all cells on the coverslip, while an MOI of 100 results in stronger staining.

FIG. 8. Expression of scleraxis in COS-7 cells infected with AdSex. COS-7 cells were infected with an adenovirus encoding HA- and FLAG-tagged scleraxis at a multiplicity of infection of 100. After 24 hours, cells were harvested and lysates prepared as described, then the lysates were run on western blots¹. Westerns were incubated with anti-HA antibodies to detect recombinant scleraxis. Lane 1 is a broad range protein standard; lanes 2 to 5 represent replicates of four different infected COS-7 cell samples. The strong lower band (˜30 kDa) represents recombinant scleraxis; the weaker upper band (˜60 kDa) likely represents minor dimer formation. Non-infected cells showed no immunoreactivity (results not shown). The 30 kDa band is identical to that previously reported for recombinant scleraxis².

FIG. 9. Infection of rat cardiac myofibroblasts with AdScx. P2 adult rat cardiac myofibroblasts were infected with adenovirus encoding HA-tagged scleraxis (AdScx) at MOI 100. Twenty-four hours after infection, scleraxis was visualized using primary rabbit anti-HA antibodies and goat anti-rabbit secondary antibodies conjugated to Texas Red. Controls were not infected with AdScx. Results are representative of two individual experiments.

FIG. 10. shRNA-mediated knockdown of AKAP84-GFP fusion protein expression. COS-7 cells were transfected with an expression vector encoding an AKAP84-GFP fusion protein alone (upper left panel) or with an shRNA vector specific to AKAP84 (upper right panel). Significant knockdown of AKAP84-GFP, noted by the reduced fluorescence, was achieved. Results are representative of at least 23 fields each. These data are quantified in the lower panel. Treatment of cells with shRNA specific to AKAP84 resulted in 80% repression of expression of co-transfected AKAP84-GFP. Mean±standard error. *p<0.0001 vs. AKAP84-GFP by Student t-test.

### DETAILED DESCRIPTION OF THE INVENTION

I. Modulation of Collagen Synthesis

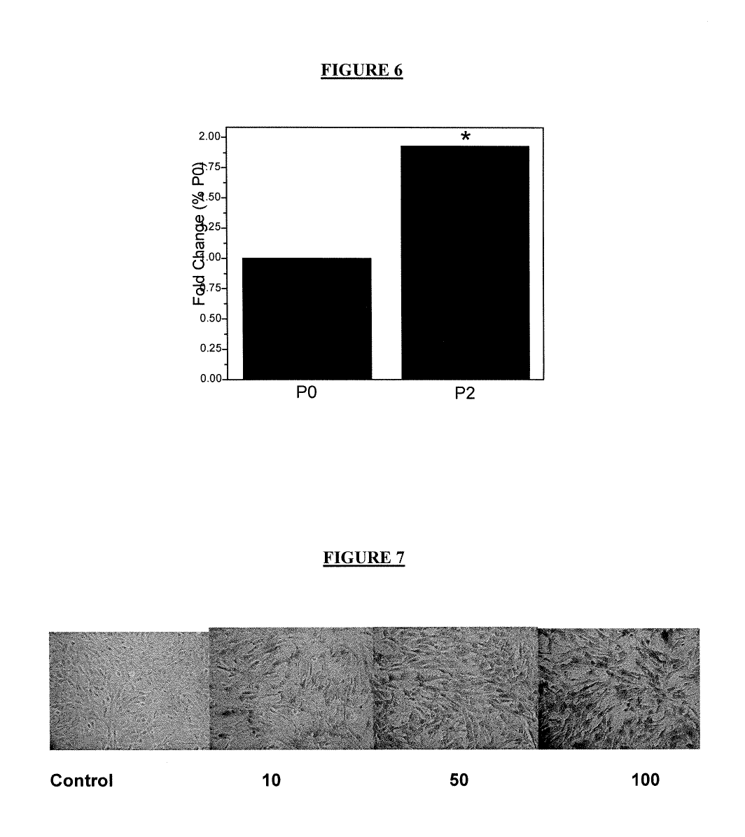

As previously stated, the present inventor has demonstrated that scleraxis is involved in collagen synthesis. Specifically, the inventor has generated experimental data showing that scleraxis can directly activate expression of a collagen Iα2 gene promoter-containing reporter vector. The result support that scleraxis can directly regulate expression of collagen genes involved in cardiac fibrosis (and likely fibrosis in other tissues as well, including kidney and skin). In contrast, a related transcription factor, MyoD, had no effect on the reporter, indicating that the effect of scleraxis is specific. The inventor has data indicating that as cardiac fibroblasts, which make very little collagen, become myofibroblasts, which make large amounts of collagen, the level of scleraxis expression in the cells increases substantially, supporting a role for scleraxis in collagen synthesis (FIG. 6; Table III).

Accordingly, the present invention provides a method of modulating collagen synthesis by administering an effective amount of an agent that modulates scleraxis to a cell or animal in need thereof. The present invention also provides a use of an effective amount of an agent that modulates scleraxis to modulate collagen synthesis. The present invention further provides a use of an effective amount of an agent that modulates scleraxis for the manufacture of a medicament to modulate collagen synthesis.

The term “scleraxis” as used herein refers to a transcription factor with a basic helix-loop-helix (bHLH) motif and includes any form of scleraxis from any species as well as analogs and homologs thereof. In one embodiment, scleraxis has the sequence shown in NCBI Accession #NM-198885 or BB474540.

The term “agent that modulates scleraxis” includes any agent that can stimulate or activate scleraxis (e.g. scleraxis agonists) as well as any agent that can inhibit or suppress scleraxis (e.g. scleraxis antagonists). Specific examples of scleraxis modulators are given in Section II.

The term “modulate collagen synthesis” as used herein refers to the inhibition or suppression as well as the activation or stimulation of the formation or development of collagen.

The term “effective amount” as used herein means an amount effective, at dosages and for periods of time necessary to achieve the desired results (e.g. the modulation of collagen synthesis). Effective amounts of a molecule may vary according to factors such as the disease state, age, sex, weight of the animal. Dosage regima may be adjusted to provide the optimum therapeutic response. For example, several divided doses may be administered daily or the dose may be proportionally reduced as indicated by the exigencies of the therapeutic situation.

The term “animal” as used herein includes all members of the animal kingdom, preferably humans.

The term “a cell” includes a single cell as well as a plurality or population of cells. Administering an agent to a cell includes both in vitro and in vivo administrations.

In one aspect, the present invention provides a method of inhibiting collagen synthesis comprising administering an effective amount of a scleraxis antagonist to a cell or animal in need thereof. The present invention also includes a use of a scleraxis antagonist to inhibit collagen synthesis or for the manufacture of a medicament to inhibit collagen synthesis.

The scleraxis antagonist can be any agent that can block the activation, stimulation or binding of scleraxis such as an antibody or antibody fragment, small molecule, peptide mimetic, peptide or an antisense oligonucleotide to scleraxis or to a molecule or sequence to which scleraxis binds.

As described in the Examples, scleraxis activates the collagen Iα2 promoter. The inventor identified 12 E-boxes in the promoter sequence of collagen Iα2. Therefore, in one embodiment, the scleraxis antagonist interferes with or prevents the binding of scleraxis to a sequence in the promoter region of collagen Iα2, preferably an E-box sequence shown in Table II. In a specific embodiment, the antagonist prevents the binding of scleraxis to: the sequence CATGTG found at nucleotide position −3317; the sequence CAGGTG found at nucleotide position −847; or the sequence CAGGTG found a nucleotide position −476, relative to the 3′ of the promoter sequence which is at −80 relative to the ATG start codon of collagen Iα2.

There are many conditions wherein one might want to prevent collagen synthesis. In one embodiment, the method is used to prevent fibrosis such as cardiac fibrosis as well as fibrosis of other tissues including kidney and skin. In a preferred embodiment, the invention provides a method of preventing or inhibiting cardiac fibrosis. The inventor has generated data demonstrating that treatment of cardiac fibroblasts with TGF-β₁, a growth factor that stimulates fibroblasts to make collagen, results in an increase in scleraxis expression. Blocking the function of scleraxis will prevent fibroblasts and myofibroblasts from making collagen; similarly, blocking the activity of scleraxis in the heart or other tissues will prevent fibrosis from occurring in these tissues. This will be important in situations where fibrosis is undesired. For example, after a heart attack, a collagen containing scar is generated to help maintain the function of the heart—this is an important healing process. However, over time, collagen continues to be synthesized in other areas of the heart (i.e. fibrosis), impairing heart function. A scleraxis blocker, given after scar formation has occurred, may prevent further fibrosis of the heart. Similar benefit may be found in other tissues in which fibrosis can occur.

In another aspect, the present invention provides a method of stimulating collagen synthesis comprising administering an effective amount of a scleraxis agonist to a cell or animal in need thereof.

The scleraxis agonist can be any agent that can stimulate scleraxis expression or activity including, but not limited to, antibodies, peptide mimetics, small molecules, scleraxis proteins and fragments thereof, and modulators identified according to the screening assays described herein. The scleraxis agonist will be useful in stimulating collagen synthesis.

Stimulation of collagen synthesis with a scleraxis agonist has utility in a wide range of therapeutic applications including any condition wherein one would want to increase the production of collagen. Examples of where increased collagen formation may be desirable would be to assist in skin/wound healing and in combating the effects of aging on the skin (e.g. improving skin tone, decreasing wrinkles). There may even be benefits to administering scleraxis to boost collagen production in skin grafts that are grown ex vivo for use in burn victims, surgery recovery, etc.

II. Scleraxis Modulators

Any agent that can modulate scleraxis and modulate collagen synthesis can be used in the methods and compositions of the invention. There are a number of different ways to alter scleraxis activity, for example, affecting its ability to interact with DNA, affecting its ability to interact with proteins (such as co-regulators), or affecting its ability to support transcription (which in some cases relates to the prior point, since transcription factors typically work by binding to the basal transcriptional machinery and recruiting it to the appropriate promoter via DNA binding). Administering a modulator that interferes with these interactions or that supports transcription antagonizes scleraxis activity. Conversely, scleraxis activity is increased by administering a modulator for stabilizing or augmenting the aforementioned interactions or for support of transcription. Some scleraxis modulators are further described below.

(a) Antibodies

Antibodies to scleraxis proteins may be used as a scleraxis modulator. Antibodies that bind to a scleraxis protein or peptide can be prepared using techniques known in the art. Conventional methods can be used to prepare the antibodies. For example, by using a peptide of a scleraxis polyclonal antisera or monoclonal antibodies can be made using standard methods. A mammal, (e.g., a mouse, hamster, or rabbit) can be immunized with an immunogenic form of the peptide which elicits an antibody response in the mammal. Techniques for conferring immunogenicity on a peptide include conjugation to carriers or other techniques well known in the art. For example, the protein or peptide can be administered in the presence of adjuvant. The progress of immunization can be monitored by detection of antibody titers in plasma or serum. Standard ELISA or other immunoassay procedures can be used with the immunogen as antigen to assess the levels of antibodies. Following immunization, antisera can be obtained and, if desired, polyclonal antibodies isolated from the sera.

To produce monoclonal antibodies, antibody producing cells (lymphocytes) can be harvested from an immunized animal and fused with myeloma cells by standard somatic cell fusion procedures thus immortalizing these cells and yielding hybridoma cells. Such techniques are well known in the art, (e.g., the hybridoma technique originally developed by Kohler and Milstein (Nature 256, 495-497 (1975)) as well as other techniques such as the human B-cell hybridoma technique (Kozbor et al., Immunol. Today 4, 72 (1983)), the EBV-hybridoma technique to produce human monoclonal antibodies (Cole et al. Monoclonal Antibodies in Cancer Therapy (1985) Allen R. Bliss, Inc., pages 77-96), and screening of combinatorial antibody libraries (Huse et al., Science 246, 1275 (1989)). Hybridoma cells can be screened immunochemically for production of antibodies specifically reactive with the peptide and the monoclonal antibodies can be isolated. Therefore, the invention also contemplates hybridoma cells secreting monoclonal antibodies with specificity for scleraxis.

The term “antibody” as used herein is intended to include fragments thereof which also specifically bind with scleraxis or peptide thereof. Antibodies can be fragmented using conventional techniques and the fragments screened for utility in the same manner as described above. For example, F(ab′)₂ fragments can be generated by treating antibody with pepsin. The resulting F(ab′)₂ fragment can be treated to reduce disulfide bridges to produce Fab′ fragments.

Chimeric antibody derivatives, i.e., antibody molecules that combine a non-human animal variable region and a human constant region are also contemplated within the scope of the invention. Chimeric antibody molecules can include, for example, the antigen binding domain from an antibody of a mouse, rat, or other species, with human constant regions. Conventional methods may be used to make chimeric antibodies containing the immunoglobulin variable region which recognizes the scleraxis protein (See, for example, Morrison et al., Proc. Natl Acad. Sci. U.S.A. 81,6851 (1985); Takeda et al., Nature 314, 452 (1985), Cabilly et al., U.S. Pat. No. 4,816,567; Boss et al., U.S. Pat. No. 4,816,397; Tanaguchi et al., European Patent Publication EP171496; European Patent Publication 0173494, United Kingdom patent GB 2177096B). It is expected that chimeric antibodies would be less immunogenic in a human subject than the corresponding non-chimeric antibody.

Monoclonal or chimeric antibodies specifically reactive with a protein of the invention as described herein can be further humanized by producing human constant region chimeras, in which parts of the variable regions, particularly the conserved framework regions of the antigen-binding domain, are of human origin and only the hypervariable regions are of non-human origin. Such immunoglobulin molecules may be made by techniques known in the art, (e.g., Teng et al., Proc. Natl. Acad. Sci. U.S.A., 80, 7308-7312 (1983); Kozbor et al., Immunology Today, 4, 7279 (1983); Olsson et al., Meth. Enzymol., 92, 3-16 (1982)), and PCT Publication WO92/06193 or EP 0239400). Humanized antibodies can also be commercially produced (Scotgen Limited, 2 Holly Road, Twickenham, Middlesex, Great Britain.)

Specific antibodies, or antibody fragments, reactive against scleraxis proteins may also be generated by screening expression libraries encoding immunoglobulin genes, or portions thereof, expressed in bacteria with peptides produced from the nucleic acid molecules of scleraxis. For example, complete Fab fragments, VH regions and FV regions can be expressed in bacteria using phage expression libraries (See for example Ward et al., Nature 341, 544-546: (1989); Huse et al., Science 246, 1275-1281 (1989); and McCafferty et al. Nature 348, 552-554 (1990)). Alternatively, a SCID-hu mouse, for example the model developed by Genpharm, can be used to produce antibodies or fragments thereof.

(b) Antisense Oligonucleotides

Antisense oligonucleotides that can modulate the expression and/or activity of scleraxis may also be used as a scleraxis antagonist. In addition, antisense oligonucleotides that can inhibit the target of scleraxis activity or binding may also be used.

The term “antisense oligonucleotide” as used herein means a nucleotide sequence that is complementary to its target.

The term “oligonucleotide” refers to an oligomer or polymer of nucleotide or nucleoside monomers consisting of naturally occurring bases, sugars, and intersugar (backbone) linkages. The term also includes modified or substituted oligomers comprising non-naturally occurring monomers or portions thereof, which function similarly. Such modified or substituted oligonucleotides may be preferred over naturally occurring forms because of properties such as enhanced cellular uptake, or increased stability in the presence of nucleases. The term also includes chimeric oligonucleotides which contain two or more chemically distinct regions. For example, chimeric oligonucleotides may contain at least one region of modified nucleotides that confer beneficial properties (e.g. increased nuclease resistance, increased uptake into cells), or two or more oligonucleotides of the invention may be joined to form a chimeric oligonucleotide.

The antisense oligonucleotides of the present invention may be ribonucleic or deoxyribonucleic acids and may contain naturally occurring bases including adenine, guanine, cytosine, thymidine and uracil. The oligonucleotides may also contain modified bases such as xanthine, hypoxanthine, 2-aminoadenine, 6-methyl, 2-propyl and other alkyl adenines, 5-halo uracil, 5-halo cytosine, 6-aza uracil, 6-aza cytosine and 6-aza thymine, pseudo uracil, 4-thiouracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thiolalkyl guanines, 8-hydroxyl guanine and other 8-substituted guanines, other aza and deaza uracils, thymidines, cytosines, adenines, or guanines, 5-trifluoromethyl uracil and 5-trifluoro cytosine.

Other antisense oligonucleotides of the invention may contain modified phosphorous, oxygen heteroatoms in the phosphate backbone, short chain alkyl or cycloalkyl intersugar linkages or short chain heteroatomic or heterocyclic intersugar linkages. For example, the antisense oligonucleotides may contain phosphorothioates, phosphotriesters, methyl phosphonates, and phosphorodithioates. In an embodiment of the invention there are phosphorothioate bonds links between the four to six 3′-terminus bases. In another embodiment phosphorothioate bonds link all the nucleotides.

The antisense oligonucleotides of the invention may also comprise nucleotide analogs that may be better suited as therapeutic or experimental reagents. An example of an oligonucleotide analogue is a peptide nucleic acid (PNA) wherein the deoxyribose (or ribose) phosphate backbone in the DNA (or RNA), is replaced with a polyamide backbone which is similar to that found in peptides (P. E. Nielsen, et al Science 1991, 254, 1497). PNA analogues have been shown to be resistant to degradation by enzymes and to have extended lives in vivo and in vitro. PNAs also bind stronger to a complementary DNA sequence due to the lack of charge repulsion between the PNA strand and the DNA strand. Other oligonucleotides may contain nucleotides containing polymer backbones, cyclic backbones, or acyclic backbones. For example, the nucleotides may have morpholino backbone structures (U.S. Pat. No. 5,034,506). Oligonucleotides may also contain groups such as reporter groups, a group for improving the pharmacokinetic properties of an oligonucleotide, or a group for improving the pharmacodynamic properties of an antisense oligonucleotide. Antisense oligonucleotides may also have sugar mimetics.

The antisense nucleic acid molecules may be constructed using chemical synthesis and enzymatic ligation reactions using procedures known in the art. The antisense nucleic acid molecules of the invention or a fragment thereof, may be chemically synthesized using naturally occurring nucleotides or variously modified nucleotides designed to increase the biological stability of the molecules or to increase the physical stability of the duplex formed with mRNA or the native gene e.g. phosphorothioate derivatives and acridine substituted nucleotides. The antisense sequences may be produced biologically using an expression vector introduced into cells in the form of a recombinant plasmid, phagemid or attenuated virus in which antisense sequences are produced under the control of a high efficiency regulatory region, the activity of which may be determined by the cell type into which the vector is introduced.

The antisense oligonucleotides may be introduced into tissues or cells using techniques in the art including vectors (retroviral vectors, adenoviral vectors and DNA virus vectors) or physical techniques such as microinjection. The antisense oligonucleotides may be directly administered in vivo or may be used to transfect cells in vitro which are then administered in vivo. In one embodiment, the antisense oligonucleotide may be delivered to macrophages and/or endothelial cells in a liposome formulation.

(c) Scleraxis

A scleraxis protein may be used as a scleraxis modulator. The scleraxis protein may be wild type or may be modified to contain amino acid substitutions, insertions and/or deletions that do not alter the ability of the peptide to modulate collagen synthesis. Conserved amino acid substitutions involve replacing one or more amino acids of the amino acid sequence with amino acids of similar charge, size, and/or hydrophobicity characteristics. When only conserved substitutions are made the resulting analog should be functionally equivalent to the scleraxis. Non-conserved substitutions involve replacing one or more amino acids of the amino acid sequence with one or more amino acids which possess dissimilar charge, size, and/or hydrophobicity characteristics.

(d) Peptide Mimetics

Peptide mimetics of scleraxis may also be prepared as a scleraxis modulator. Such peptides may include competitive inhibitors, enhancers, peptide mimetics, and the like. All of these peptides as well as molecules substantially homologous, complementary or otherwise functionally or structurally equivalent to these peptides may be used for purposes of the present invention.

“Peptide mimetics” are structures which serve as substitutes for peptides in interactions between molecules (See Morgan et al (1989), Ann. Reports Med. Chem. 24:243-252 for a review). Peptide mimetics include synthetic structures which may or may not contain amino acids and/or peptide bonds but retain the structural and functional features of a peptide, or enhancer or inhibitor of the invention. Peptide mimetics also include peptoids, oligopeptoids (Simon et al (1972) Proc. Natl. Acad. Sci. USA 89:9367); and peptide libraries containing peptides of a designed length representing all possible sequences of amino acids corresponding to a peptide of the invention.

Peptide mimetics may be designed based on information obtained by systematic replacement of L-amino acids by D-amino acids, replacement of side chains with groups having different electronic properties, and by systematic replacement of peptide bonds with amide bond replacements. Local conformational constraints can also be introduced to determine conformational requirements for activity of a candidate peptide mimetic. The mimetics may include isosteric amide bonds, or D-amino acids to stabilize or promote reverse turn conformations and to help stabilize the molecule. Cyclic amino acid analogues may be used to constrain amino acid residues to particular conformational states. The mimetics can also include mimics of inhibitor peptide secondary structures. These structures can model the 3-dimensional orientation of amino acid residues into the known secondary conformations of proteins. Peptoids may also be used which are oligomers of N-substituted amino acids and can be used as motifs for the generation of chemically diverse libraries of novel molecules.

Peptides derived from scleraxis isoforms may also be used to identify lead compounds for drug development. The structure of the peptides described herein can be readily determined by a number of methods such as NMR and X-ray crystallography. A comparison of the structures of peptides similar in sequence, but differing in the biological activities they elicit in target molecules can provide information about the structure-activity relationship of the target. Information obtained from the examination of structure-activity relationships can be used to design either modified peptides, or other small molecules or lead compounds that can be tested for predicted properties as related to the target molecule. The activity of the lead compounds can be evaluated using assays similar to those described herein.

Information about structure-activity relationships may also be obtained from co-crystallization studies. In these studies, a peptide with a desired activity is crystallized in association with a target molecule, and the X-ray structure of the complex is determined. The structure can then be compared to the structure of the target molecule in its native state, and information from such a comparison may be used to design compounds.

(e) Screening Assays

The present invention also includes screening assays for identifying agents that modulate scleraxis and that are useful in modulating collagen synthesis.

In accordance with one embodiment, the invention enables a method for screening candidate compounds for their ability to modulate the activity of scleraxis.

Accordingly, the present invention provides a method for identifying a compound that modulates collagen synthesis comprising:

(a) incubating a test compound with a cell expressing scleraxis; and

(b) determining the effect of the compound on scleraxis activity or expression and comparing with a control (i.e. in the absence of the test compound), wherein a change in the scleraxis activity or expression as compared to the control indicates that the test compound may modulate collagen synthesis.

In one embodiment, the screening assay can be used to identify scleraxis antagonists.

Accordingly, the present invention provides a screening assay for identifying an antagonist of scleraxis useful in inhibiting collagen synthesis comprising the steps of:

(a) incubating a test compound with scleraxis; and

(b) determining whether or not the test compound inhibits scleraxis, wherein inhibition of scleraxis indicates that the compound is a scleraxis antagonist and may be useful in inhibiting collagen synthesis.

In all of the above screening assays, the test compound can be any compound which one wishes to test including, but not limited to, proteins, peptides, nucleic acids (including RNA, DNA, antisense oligonucleotide, peptide nucleic acids), carbohydrates, organic compounds, small molecules, natural products, library extracts, bodily fluids and other samples that one wishes to test for modulators of scleraxis.

The cell used in the screening assay can be any cell that expresses scleraxis or a cell that has been transfected with scleraxis. Types of cells that may be used include, but are not limited to, COS-7 or 293 cell lines. In a specific embodiment, the cells also express a scleraxis-sensitive reporter gene (e.g. the collagen Iα2 promoter) that drives the expression of a detectable product such as green fluorescent protein which can be detected on a fluorometric plate reader. A potentially useful scleraxis antagonist would be expected to reduce the fluorescent signal measured.

The screening methods of the invention include high-throughput screening applications. For example, a high-throughput screening assay may be used which comprises any of the methods according to the invention wherein aliquots of cells transfected with scleraxis are exposed to a plurality of test compounds within different wells of a multi-well plate. Further, a high-throughput screening assay according to the invention involves aliquots of transfected cells which are exposed to a plurality of candidate factors in a miniaturized assay system of any kind. Another embodiment of a high-throughput screening assay could involve exposing a transfected cell population simultaneously to a plurality of test compounds.

The method of the invention may be “miniaturized” in an assay system through any acceptable method of miniaturization, including but not limited to multi-well plates, such as 24, 48, 96 or 384-wells per plate, micro-chips or slides. The assay may be reduced in size to be conducted on a micro-chip support, advantageously involving smaller amounts of reagent and other materials. Any miniaturization of the process which is conducive to high-throughput screening is within the scope of the invention.

The invention extends to any compounds or modulators of scleraxis identified using the screening method of the invention that are useful in modulating collagen synthesis.

The invention also includes a pharmaceutical composition comprising a modulator of scleraxis identified using the screening method of the invention in admixture with a suitable diluent or carrier. The invention further includes a method of preparing a pharmaceutical composition for use in modulating collagen synthesis comprising mixing a modulator of scleraxis identified according to the screening assay of the invention with a suitable diluent or carrier.

III. Pharmaceutical Compositions

The present invention includes pharmaceutical compositions containing one or more modulators of scleraxis. Accordingly, the present invention provides a pharmaceutical composition comprising an effective amount of a scleraxis modulator in admixture with a suitable diluent or carrier.

In one embodiment, the present invention provides a pharmaceutical composition for use in inhibiting collagen synthesis comprising an effective amount of a scleraxis antagonist in admixture with a suitable diluent or carrier.

Such pharmaceutical compositions can be for intralesional, intravenous, topical, rectal, parenteral, local, inhalant or subcutaneous, intradermal, intramuscular, intrathecal, transperitoneal, oral, and intracerebral use. The composition can be in liquid, solid or semisolid form, for example pills, tablets, creams, gelatin capsules, capsules, suppositories, soft gelatin capsules, gels, membranes, tubelets, solutions or suspensions. The scleraxis modulator is preferably injected in a saline solution either intravenously, intraperitoneally or subcutaneously.

The pharmaceutical compositions of the invention can be intended for administration to humans or animals. Dosages to be administered depend on individual needs, on the desired effect and on the chosen route of administration.

The pharmaceutical compositions can be prepared by per se known methods for the preparation of pharmaceutically acceptable compositions which can be administered to patients, and such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985).

On this basis, the pharmaceutical compositions include, albeit not exclusively, the active compound or substance in association with one or more pharmaceutically acceptable vehicles or diluents, and contained in buffered solutions with a suitable pH and iso-osmotic with the physiological fluids. The pharmaceutical compositions may additionally contain other immune modulatory agents.

The following non-limiting examples are illustrative of the present invention:

### EXAMPLE

### Examples

### Scleraxis Regulates Collagen Expression In Cardiac Myofibroblasts.

I) Experiments:

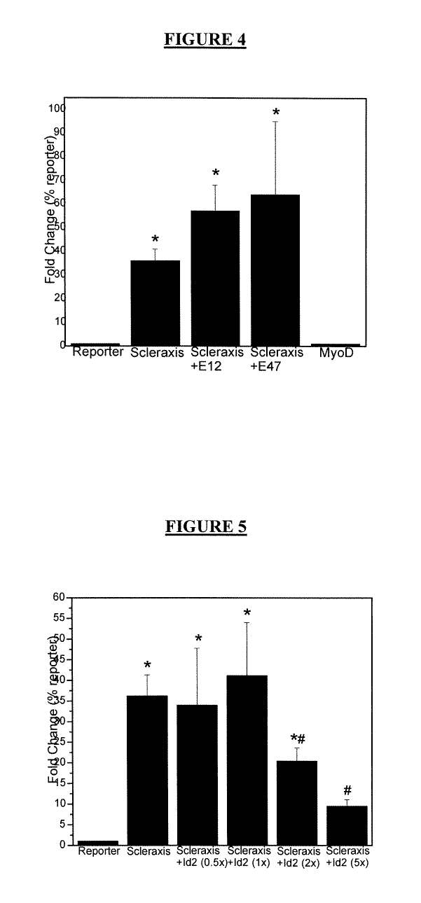

1. To demonstrate that scleraxis is sufficient to transactivate the collagen Iα2 gene. This collagen is a primary constituent of the ECM of the heart and the most significant contributor to myocardial fibrosis in the diseased heart⁵. The inventor will perform transient transfections of COS-7 cells with a mouse scleraxis expression vector and a luciferase reporter under control of 3.7 kb of the collagen type Iα2 gene promoter. Preliminary data demonstrates that scleraxis strongly activates this reporter (FIG. 4). Examination of the promoter sequence reveals the presence of 12 potential E-boxes to which scleraxis may bind (Table II). The inventor will therefore perform EMSAs and deletion analyses in order to determine which of the E-boxes are responsible/necessary for the effect of scleraxis on expression of the reporter. The inventor will also examine whether scleraxis induces collagen expression in cardiac myofibroblasts. Myofibroblasts will be infected with an adenovirus encoding scleraxis, then assayed for collagen synthesis and expression using histological staining, western blotting and a collagen assay kit.

2. To examine the role of scleraxis in collagen expression by cardiac myofibroblasts in vitro. The inventor will stimulate collagen production in cardiac myofibroblasts, the primary source of collagen synthesis in the diseased heart, using TGF-β₁, or angiotensin II, then examine whether scleraxis expression is induced using RT-PCR, and western blotting or immunocytochemistry with anti-scleraxis antibodies. The inventor will then determine whether scleraxis induction is necessary for collagen gene up-regulation by generating shRNA packaged into an adenoviral vector in order to knockdown scleraxis expression. This adenovirus will be used to infect cardiac myofibroblasts, which will then be stimulated with TGF-β₁, or angiotensin II. Collagen production will be assessed as in Experiment 1.

3. To demonstrate that scleraxis expression is induced in an animal model of cardiac fibrosis in vivo. The inventor will induce myocardial infarction (MI) in adult rat hearts by ligation of the left anterior descending coronary artery. At four time-points after infarct, the inventor will examine expression of scleraxis by RT-PCR of myofibroblast total RNA, and by immunofluorescence of cardiac tissue sections, and will correlate this with collagen expression and proximity to the infarct region detected by histological staining.

Together, the experiments described here will elucidate the role of the bHLH transcription factor scleraxis in the synthesis of collagen by cardiac myofibroblasts both in vitro and in vivo. These studies will indicate whether scleraxis is sufficient for inducing collagen production by testing the effect of scleraxis on a collagen promoter reporter. The inventor will also examine whether scleraxis is necessary for collagen production by testing whether scleraxis is induced in response to well-characterized triggers of fibrosis, and whether inhibition of scleraxis expression by RNAi can prevent collagen synthesis in response to these triggers. Finally, the inventor will examine whether scleraxis expression is correlated with fibrosis in a rat infarct model. These studies will improve the understanding of how collagen synthesis is controlled at the transcriptional level and will confirm scleraxis as a novel and important mediator of fibrosis and potential therapeutic target.

II) Introduction

A. Cardiac Fibrosis and the Role of Myofibroblasts

In response to a prolonged increase in workload or stress, the heart undergoes dramatic changes in morphology that impact cardiac function. Initial changes include hypertrophy of cardiomyocytes, resulting in an increase in muscularity of the heart⁶. These changes are thought to be adaptive in nature, and may represent an attempt to normalize the workload of the heart. After myocardial infarct, for example, the ventricles increase in volume and significant hypertrophy occurs, which may reflect an attempt to improve stroke volume following loss of viable tissue in the infarct scar region⁶. Over time, these morphological changes often lead to decompensation, which is marked by thinning of the myocardium, loss of cardiomyocytes, generalized fibrosis and a devolution of cardiac contractile function into overt heart failure, resulting in significant morbidity and mortality of cardiac patients^{6,7}.

During the shift from compensated to decompensated hypertrophy, cardiac fibrosis occurs gradually, whereby ECM, particularly collagens, are synthesized throughout the heart⁶. In contrast, following an MI, matrix production is significantly increased in the region of the infarct almost immediately. Within three weeks, the infarct scar is well-established, but further maturation and thinning of the scar continues thereafter⁸. This scar formation is an important reparative process that strengthens the weakened myocardium in the region of infarct and prevents aneurysm, and in which migration of myofibroblasts to the scar is critical⁹. However, fibrosis also occurs in regions of the heart distal to the MI itself, as cardiac function is impaired and generalized hypertrophy occurs^{10,11}. Infarct size correlates directly with the impact on the function of the remaining viable myocardium: smaller infarcts result in cardiac hypertrophy, while larger infarcts can lead directly to cardiac failure¹²⁻¹⁴.

In the healthy heart, fibroblasts are abundant while myofibroblasts are primarily associated with the valves¹⁵. During fibrosis, many fibroblasts shift to the myofibroblast phenotype, the primary source of ECM synthesis in cardiac pathology¹⁶⁻¹⁸. Alternatively, myofibroblasts can be recruited from circulating myofibroblast progenitors⁹. Following infarct, myofibroblasts are recruited to the scar, where they can persist for many years¹⁹. Myofibroblasts express markers that distinguish them from fibroblasts (α-smooth muscle actin (α-SMA) and embryonic smooth muscle myosin heavy chain (SM_{emb})), and that affect how they function in the heart, e.g. AT₁ receptors, TGF-β₁ receptors and a variety of collagens^{9,20-24}. TGF-β₁ plays a key role in processes such as cell growth and differentiation, tissue wound repair and ECM production²⁵⁻²⁷. Activation of myofibroblasts in the heart by local production of factors such as angiotensin II or TGF-β₁, results in synthesis of collagens and other molecules involved in the fibrotic response²⁸⁻³². The signaling pathways activated by TGF-β₁ or angiotensin II ligand binding have been extensively studied. For example, a variety of Smads have been implicated in TGF-β₁ regulation of collagen synthesis and the activation of myofibroblasts^{21,33}. The inappropriate activation of myofibroblasts in regions of the heart distal to the infarct scar may play a key role in loss of cardiac compliance, thus myofibroblast activation may have both beneficial (i.e. scar formation) and pathological consequences for the myocardium^{9,11}. The transcription factor effectors of signaling pathways involved in collagen synthesis by activated myofibroblasts are incompletely characterized. The bHLH transcription factor scleraxis plays a role in regulating gene expression of connective tissue genes, such as collagen II, in other tissues, but the potential role of scleraxis in cardiac remodeling and myofibroblast function has not been investigated.

B. Scleraxis

i) Function and Expression of Scleraxis:

Scleraxis was originally cloned in a yeast two-hybrid screen for interacting partners of the ubiquitous bHLH transcription factor E12². Basic helix-loop-helix transcription factors bind specifically to DNA consensus sequences called E-boxes, which have the sequence CANNTG, where N is any nucleotide^{2,34}. Different E-box binding factors appear to have distinct relative preferences for different classes of E-boxes, depending on the identity of the two internal nucleotides⁴. In the initial report of its cloning, scleraxis was demonstrated to bind specifically to E-boxes, and to drive transcription of a reporter gene under control of an artificial promoter construct containing four E-boxes². E12 augmented the ability of scleraxis to bind to oligonucleotides encoding E-boxes derived from the muscle creatine kinase gene promoter. Another E-box binding transcription factor, E47, has also been demonstrated to bind to scleraxis. Like E12, the interaction with E47 augmented the ability of scleraxis to bind to E-boxes, however, these experiments utilized artificial promoter/reporter gene constructs, thus the dependency of scleraxis activity on E proteins is unclear³⁵. The Noda laboratory identified the first gene whose expression is directly regulated by scleraxis, aggrecan, and noted that scleraxis was able to drive expression of an aggrecan promoter reporter construct without the addition of exogenous E-box binding factors such as E12 or E47⁴. It appears, therefore, that the dependence of scleraxis on heterodimerization with E-box binding factors for full transactivation activity is context-sensitive, likely depending on the specific sequence of the E-box or the particular cell type involved⁴.

During mouse embryonic development, scleraxis is widely-expressed at the time of gastrulation around embryonic day 6.0, but soon after becomes restricted in its expression pattern³⁶. Scleraxis is expressed at least as early as embryonic day 9.5 in the sclerotome compartment of somites, from which the ribs and vertebrae arise². High expression of scleraxis is noted in a number of pre-skeletal mesenchymal cells prior to chondrogenesis, but expression levels decrease during ossification. Notably, scleraxis is also highly expressed in cells that form tendons, ligaments and bronchial cartilage, as well as throughout the pericardium². Mouse embryos lacking the scleraxis gene fail to gastrulate at embryonic day 6.0. Experiments with scleraxis-null and wild type chimeric embryos revealed that cells lacking scleraxis failed to populate the sclerotome, yet were able to contribute to most other embryonic tissues, demonstrating a critical role for scleraxis in the development of sclerotome-derived tissues³⁶. Recently, Yutzey et al. demonstrated that scleraxis is expressed in the developing chordae tendinae proximal to the papillary muscles of embryonic chick hearts³.

The expression pattern of scleraxis suggests involvement in specification and maturation of tissues rich in connective tissue such as tendons and cartilage³⁷. Recent experiments demonstrate that scleraxis-positive cells form a distinct fourth compartment of the somite during embryogenesis, called the “syndetome,” distinct from the myotome, dermatome and sclerotome regions^{38,39}. These cells give rise to tendons associated with the axial skeleton, and arise spatially between muscle and cartilage progenitors. Recent evidence suggests that tendons and cartilage are alternate fates of a common cell progenitor⁴⁰. Expression of scleraxis also occurs in the interface region between muscles and skeletal primordia in 13.5 day old mouse embryos, and becomes largely restricted to tendons by 15.5 days⁴¹.

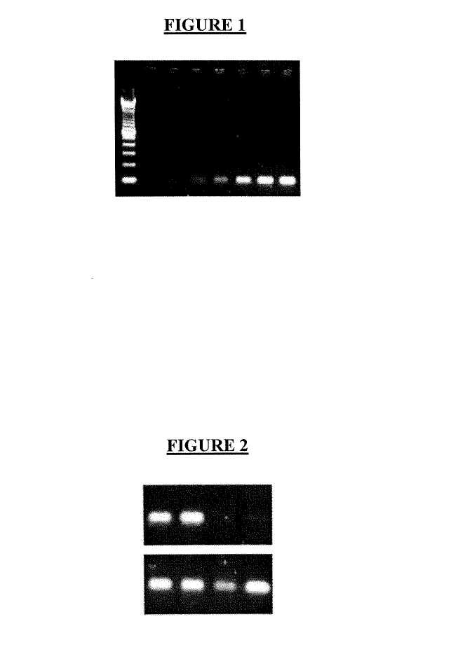

Scleraxis' role in the heart is largely unknown, although it has been cloned out of an embryonic heart gene expression library (NCBI acc. #BB474540). As mentioned above, scleraxis appears to play a role in modeling cardiac valve structures, i.e. chordae tendinae³, but the cited study examined very limited time points in valvulogenesis, therefore it is unclear whether scleraxis may have a larger role to play in cardiac development, or even at other points of valve morphogenesis. Commercial antibodies for scleraxis are not yet available, therefore the inventor performed RT-PCR of total RNA isolated from several cell types using primers specific to scleraxis (primer sequences are given in Experiment 2, below; FIGS. 1 and 2). Expression of scleraxis was observed in both isolated cardiomyocytes and isolated cardiac P2 myofibroblasts. In contrast, scleraxis showed almost no expression in either rat liver or in COS-7 cells. Others have reported expression in the heart using total cardiac RNA (and lack of expression in liver), but did not examine the relative expression in cardiomyocytes and myofibroblasts⁴².

ii) Gene Targets of Scleraxis:

The transcriptional gene targets of scleraxis are largely unknown. Scleraxis is important for Sertoli cell function in the testis, where it regulates the expression of transferrin and androgen binding protein⁴². The only other gene conclusively demonstrated to be directly regulated at the transcriptional level by scleraxis is aggrecan 1, a major proteoglycan component of cartilage, which is upregulated in response to scleraxis overexpression in ROS17/2.8 osteoblastic osteosarcoma cells^{4,43}. Scleraxis overexpression resulted in increased expression of the cartilage markers collagen II and osteopontin, and decreased expression of the osteoblast phenotype markers collagen I and alkaline phosphatase, but the mechanism of these expression changes was not determined⁴. During development of heart valves, scleraxis expression is correlated with expression of collagen II and tenascin³. Similarly, differentiation of embryonic stem cells to a chondrocyte phenotype is marked by coordinated upregulation of scleraxis, collagen IIb and aggrecan⁴⁴. While scleraxis overexpression was associated with a decrease in collagen I expression in ROS17/2.8 cells, both genes were coordinately upregulated in a pluripotent tendon-derived cell line⁴⁵. These studies in different tissues and cell lines suggest that the target genes of scleraxis are context-sensitive, resulting in different roles for scleraxis in different environments. Some genes (e.g. collagen II) may show differential regulation by scleraxis depending on cell type. The inventor has performed experiments suggesting that scleraxis regulates fibrillar collagen synthesis in the heart.

The inventor previously generated a transgenic mouse line in which a mutant form of histone deacetylase 5, a constitutive repressor of the muscle-enriched transcription factor family MEF2, is overexpressed in the heart under control of the drug doxycycline (DOX)¹. When DOX is removed from the water supply of these animals, the transgene is induced, with measurable levels of transgene expression first detected approximately four days later. Within ten days after DOX withdrawal, virtually all male animals die from apparent acute cardiac failure, due primarily to defects in mitochondrial structure and function, and mass downregulation of energy-generating metabolic genes¹. The inventor performed DNA microarray analysis of total cardiac cDNA in these animals and noted increased expression of genes associated with cardiac hypertrophy and failure, including ANF, BNP and MCARP. The inventor was intrigued to find that, at both one and four days after full transgene activation, there was a strong induction of scleraxis, from 2.5 to 3.5-fold over the expression level in healthy control hearts (Table I). These increases were highly statistically significant (p<0.0001). The inventor also noted that expression of all collagen and procollagen genes on the microarray was normal one day after full transgene activation. After four days of transgene expression, several collagen genes associated with cardiac fibrosis were strongly upregulated, including collagen Iα1, Iα2, IIIα1 and Vα2 (Table I). These collagen species represent the major constituents of cardiac fibrotic scar following myocardial infarct: collagen I accounts for approximately 40% of scar collagen content, with collagens III and V accounting for 35% and 25%, respectively⁵. Conversely, the inventor saw no change in the expression of collagens not typically involved in fibrosis, such as collagen IVα1, IVα2, VIα1, VIα2, XV and numerous others. Collagens IIα1, IXα2, and XIα1, which are all primary components of cartilage, showed no change in expression (Table I)⁴⁶.

In light of the striking temporal relationship between scleraxis induction in these mice and the upregulation of fibrotic collagen isoforms, as well as experiments associating scleraxis expression changes with collagen expression changes in other tissues, the inventor hypothesized that scleraxis may be a direct regulator of cardiac collagen gene expression, and that scleraxis induction during cardiac dysfunction may contribute to fibrosis. In support of this idea, it is intriguing to note that TGF-β₁, which can stimulate scleraxis expression in osteoblasts⁴⁷, is one of the best-characterized inducers of cardiac fibrosis, although the transcription factor effectors of this process are largely unknown⁵. The inventor therefore looked for putative E-boxes in the sequence of the promoter for collagen Iα2, the primary collagen expressed in cardiac fibrosis⁵. Within a 3.7 kb region spanning from −3800 to −80 relative to the ATG start codon, the inventor identified 12 putative E-boxes, of which three match sequences known to bind scleraxis with high affinity (Table II)^{2,4}. It is therefore possible that scleraxis may transactivate this promoter. To directly examine this possibility, the inventor transfected a reporter gene containing this 3.7 kb promoter region driving expression of luciferase (ColIα2-3.7 kb/pGL3-Luc, cloned by Steve Jones in the laboratory of Dr. I. Dixon) into COS-7 cells using Lipofectamine 2000 (Invitrogen), with or without an HA and FLAG-tagged scleraxis mammalian expression vector (pECE-FLAG HA-Scx, courtesy of Dr. E. Olson)². Luciferase activity was assayed using a luciferase reporter assay system (Promega), and was normalized to co-transfected β-galactosidase activity¹. The data in FIG. 4 demonstrates that scleraxis potently up-regulates expression of this reporter construct by 36-fold.

The inventor also examined the effects of E12 or E47 bHLH transcription factors co-transfected with scleraxis: there was a small but non-significant increase in expression of the collagen Iα2 reporter gene relative to scleraxis alone. This limited increase may reflect the presence of endogenous E proteins in COS-7 cells. In the preliminary experiments, the inventor could significantly attenuate scleraxis transactivation of the reporter by co-transfection of Id2 (pCMV-Id2, courtesy of Dr. L. Kirshenbaum) in a dose-dependent manner (FIG. 5). Id2 structurally resembles other bHLH protein family members, but lacks the basic region that mediates DNA interaction. It binds to other E-box binding proteins such as E12 and E47 and represses their activity by interfering with protein complex binding to DNA^{48,49}. The results suggest that endogenous E proteins affect the control of collagen expression by scleraxis in these assays, and may explain why exogenous E12 or E47 had no significant effect on scleraxis transactivation of the reporter gene. This is likely due to Id2 interacting directly with the E proteins, since previous reports indicate that Id2 does not bind to scleraxis⁴². The inventor also examined whether another E-box binding bHLH transcription factor, MyoD, is able to activate collagen Iα2 expression. The results demonstrate that MyoD has no effect on reporter expression, indicating that scleraxis is a specific regulator of this promoter (FIG. 4).

The preliminary experiments provide strong support that scleraxis may play a critical role in regulating expression of collagen I, a primary component of fibrosis in the heart. Furthermore, the microarray and RT-PCR data indicate that scleraxis is expressed in myofibroblasts, and cardiac scleraxis levels increase during acute heart failure. The experiments outlined in this example have been designed to further assess the role played by scleraxis in regulating fibrosis by examining three issues: 1. how scleraxis regulates collagen expression at the level of interaction with specific DNA sequence elements; 2. the role scleraxis plays in the synthesis of collagen by cardiac myofibroblasts; 3. the involvement of scleraxis in fibrosis of the heart in an animal model of MI.

III) Experiments, Design and Methodology

Experiment 1: Scleraxis is Sufficient to Transactivate the Collagen Iα2 Gene.

With the exception of the aggrecan 1, transferrin and androgen binding protein genes, the transcriptional targets of scleraxis are unknown. However, empirical evidence suggests that scleraxis may regulate the expression of collagen II⁴. Both up-regulation and down-regulation of collagen I synthesis has been reported in response to scleraxis expression, therefore it is unclear what the true role of scleraxis is in mediating collagen I expression^{4,45}. As discussed earlier, the specific targets of scleraxis, and the effect of scleraxis on transactivation of those targets (i.e. activation or repression) are likely cell-specific. The microarray data (discussed above) supports the idea that scleraxis may be a general regulator of collagen expression during fibrosis. The first experiment is therefore to demonstrate that scleraxis is sufficient to transactivate collagen Iα2 gene expression.

The preliminary data (discussed above) directly address this objective. Scleraxis potently transactivates the collagen Iα2 gene promoter, an effect that likely involves E-box binding proteins (FIGS. 4 and 5). This effect appears to be specific to scleraxis, since MyoD had no effect (FIG. 4). The inventor will extend these data further in order to further demonstrate how scleraxis regulates this promoter. The inventor will first perform more luciferase reporter assays with transfected scleraxis, increasing the amount of co-transfected Id2 similar to the experiment in FIG. 5 to see whether the effect of scleraxis can be completely attenuated. This experiment will help to determine to what degree transactivation of the collagen Iα2 promoter is due to scleraxis alone: if the inventor is unable to completely attenuate the effects of scleraxis by Id2 co-transfection, this will suggest that scleraxis does not require an E protein partner in order to drive collagen Iα2 gene expression. This result will be important, since previous reports have demonstrated that scleraxis can transactivate gene expression either independently of E12 and E47, or in conjunction with either factor^{2,4,35}. The inventor will also delete the helix-loop-helix moiety of scleraxis (amino acids 91-131) using nested PCR to abrogate interaction of scleraxis with E proteins². This mutant construct will be transfected into COS-7 cells as above to determine whether scleraxis can still transactivate the reporter. To confirm the importance of scleraxis DNA binding activity for transactivation of the reporter, the inventor will delete the DNA-binding basic region of scleraxis (amino acids 78-90)² using nested PCR and perform similar luciferase assays. It is expected that deletion of the basic region will render scleraxis unable to transactivate the promoter. All luciferase assays will be performed as described above. Statistics on results in triplicate will be carried out using one-way ANOVA or Student t-test as appropriate, with p<0.05 considered statistically significant.

The inventor will generate oligonucleotides corresponding to each E-box shown in Table II for use in EMSAs. These oligos will be labeled with α³²P-dCTP and incubated with lysates of COS-7 cells expressing HA-tagged scleraxis or empty vector as the inventor has performed previously¹. Specificity of binding will be demonstrated using competition with cold (unlabeled) oligonucleotides in 100-fold molar excess, and by comparison with identical experiments with oligos in which the E-box has been randomized; alternatively, the inventor will perform supershifts with anti-HA antibodies¹. To demonstrate the importance of different regions of the promoter for scleraxis transactivation, the inventor will delete progressively larger regions of the promoter starting from the 5′ or 3′ ends and repeat the luciferase reporter assays described above. Similarly, the inventor will also delete the region −2900 to −2700, which contains three putative E-boxes, and −500 to −450, which contains two putative E-boxes in close proximity, similar to those observed in the scleraxis-regulated aggrecan 1 gene promoter⁴. Luciferase assays of the promoter deletion constructs will be compared to the wild-type promoter results. The inventor expects that deletion of these two regions, in particular, will significantly impair the ability of scleraxis to transactivate the collagen Iα2 gene promoter.

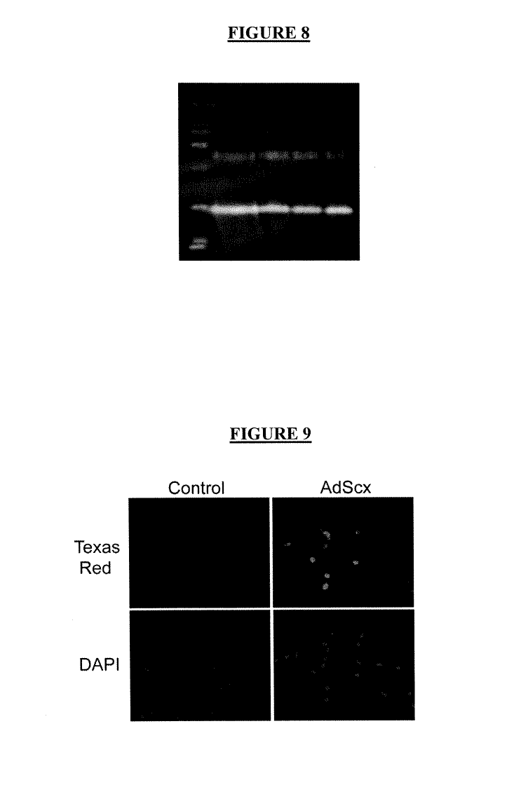

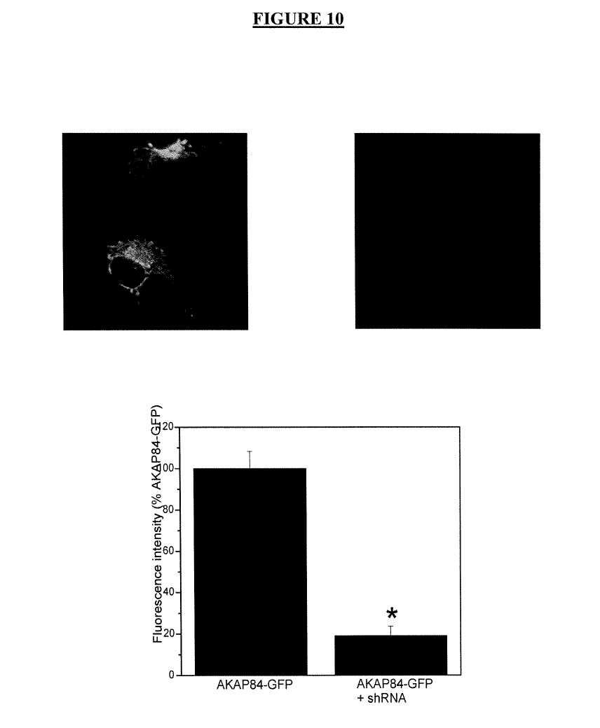

To examine whether scleraxis can induce collagen synthesis in cardiac myofibroblasts, the inventor will infect primary adult rat passage 2 (P2) myofibroblasts with an adenovirus encoding HA and FLAG-tagged scleraxis, then assay for collagen production. Passage 2 myofibroblasts most closely resemble myofibroblasts that are found in the myocardial infarct scar in vivo, with respect to expression of marker genes and ability to synthesize collagen^{9,51}. P2 myofibroblasts synthesize large amounts of collagen, are contractile, form focal adhesions and express unique marker genes compared to primary isolated cardiac fibroblasts (P0) (Table III). However, since P2 myofibroblasts already synthesize significant amounts of collagen, these experiments will be repeated with freshly isolated cardiac fibroblasts (P0). These cells will be used within 24 hours of isolation, since they gain myofibroblast character with increasing time in culture. Strikingly, the preliminary data indicate that scleraxis expression increases 100% as fibroblasts become myofibroblasts, which parallels the increase in collagen synthesis by these cells (FIG. 6). The isolation of myofibroblasts has been thoroughly described elsewhere⁵⁰. Briefly, cells are isolated using a retrograde perfusion technique from hearts of adult male Sprague-Dawley rats (200-225 g). Hearts are perfused with 0.1% collagenase (Worthington), and isolated cells collected by centrifugation. After differential plating of cells for 3 hours, non-adherent cardiomyocytes are discarded. Adherent cells (primarily fibroblasts) are cultured overnight in DMEM/F12 containing 10% FBS, 100 U/ml penicillin, 100 μg/ml streptomycin and 100 μM ascorbate, then passaged twice⁵⁰. The purity of these cells is typically >95%.⁵⁰ The generation of the scleraxis adenovirus (AdScx) was performed at the viral core facility of the University of Texas Southwestern Medical Center (courtesy of Dr. B. Gerard). The inventor is experienced in the use of adenoviruses for infection of mammalian cells,¹ and has successfully infected P2 myofibroblasts with β-galactosidase reporter adenovirus, attaining greater than 95% infection efficiency with a multiplicity of infection (MOI) of 50 (FIG. 7). The inventor has also successfully infected COS-7 cells with the AdScx adenovirus, demonstrating efficient production of scleraxis (FIG. 8), and are currently optimizing infection of myofibroblasts with this virus using different MOIs. To date, the inventor has obtained greater than 50% infection efficiency of AdScx in P2 myofibroblasts, and will improve efficiency by plaque-purification of the adenovirus (FIG. 9). The inventor will infect P2 myofibroblasts with AdScx at an MOI of 10, 50, 100 or 150 in order to determine optimal efficiency of infection: 24 hours after infection, cells will be fixed in 10% formalin in PBS, then incubated with anti-HA antibody (Santa Cruz), followed by FITC-labeled secondary antibody. The MOI that gives optimal efficiency will be used for all further experiments. Controls will consist of non-infected cells, or cells infected with an adenovirus encoding β-galactosidase (Ad-LacZ) (FIG. 7).¹

Infected cells will be used at 24, 48 or 72 hours after infection. Collagen synthesis by fibroblasts or myofibroblasts will be determined by picrosirius red staining of cells to demonstrate general collagen synthesis, or by western blotting of whole cell lysates using SP1.D8 antibodies, which recognize procollagen type I⁹. These two methods complement each other: picrosirius red stains for mature collagen, while SP1.D8 recognizes procollagen (i.e. immature collagen precursor), thus the inventor will be able to detect collagen synthesis regardless of how quickly or slowly it is generated and processed. The inventor will also use a commercial kit (Orion Diagnostica) to assay for procollagen-1 N-terminal propeptide (PINP) in cell media, which is a measure of mature secreted collagen⁹. The inventor will confirm scleraxis expression in these cells by western blotting with anti-HA antibody. These experiments will demonstrate whether scleraxis expression is sufficient for collagen synthesis by myofibroblasts.

Experiment 2: The Role of Scleraxis in Collagen Expression by Cardiac Myofibroblasts in Vitro.

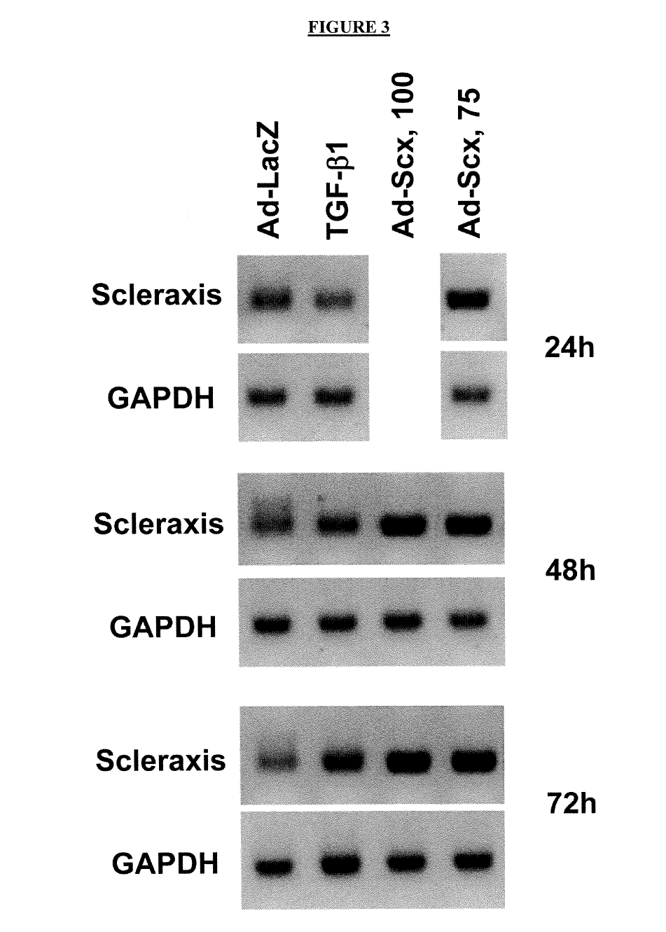

Intriguingly, osteoblasts increase scleraxis expression in response to TGF-β1⁴⁷. Myofibroblasts synthesize collagen in response to stimulation by angiotensin II or TGF-β1⁵, but it is unclear whether this response involves increased expression of scleraxis. The inventor stimulated P0 neonatal rat cardiac fibroblasts with 10 ng/ml TGF-β1, compared to infection with adenoviruses encoding scleraxis (Ad-Scx) or β-galactosidase (Ad-LacZ) and measured scleraxis expression by RT-PCR (FIG. 3). TGF-β1 induced a time-dependent increase in scleraxis expression compared to Ad-LacZ control. Ad-Scx was used as a positive control. The inventor will stimulate P2 rat myofibroblasts with 10 ng/ml TGF-β1, or will stimulate P0 fibroblasts or P2 myofibroblasts with 10⁻⁷ M angiotensin II, or vehicle. These concentrations induce collagen synthesis in myofibroblasts⁹ (I. Dixon, personal communication). At various timepoints (1, 2, 4, 6, 24, 48 or 72 hours), cells will be harvested and whole cell lysates prepared as the inventor prepared previously¹. These lysates will be run on SDS-PAGE gels and western blotted using anti-scleraxis antibodies. These antibodies will be prepared using a commercial genetic immunization DNA-to-antibody service (QED Bioscience) by first cloning the rat scleraxis cDNA in-frame into pSecTag2 (Invitrogen) to ensure proper presentation of antigen. Two rabbits will be used for antibody preparation to increase the chances of success. Antibodies will be screened by western blotting of lysates of scleraxis-transfected COS-7 cells. If the inventor is unable to generate satisfactory antibodies, the inventor will isolate total RNA from cells at each time-point using Trizol (Invitrogen) as the inventor has performed previously¹. Total cDNA will be prepared using Superscript III (Invitrogen). The inventor will perform RT-PCR of the total cDNA samples using oligonucleotide primers specific for scleraxis (forward primer:5′-ATG TCC TTC GCC ATG CTG CGT-3′; reverse primer:5′-CTA ACT TCG AAT CGC CGT CTT TCT GTC-3′). These primers encompass the entire scleraxis coding region, and span the sole intron, and thus will not prime genomic DNA⁵³. The inventor is experienced in the use of RT-PCR¹, and has successfully performed semi-quantitative RT-PCR of scleraxis using these primers (FIGS. 1, 2 and 3). The inventor recently established a SyBr green method for real-time RT-PCR in the laboratory, and is adapting the current method to work on this equipment. These experiments will demonstrate induction of scleraxis expression in response to activators of fibrosis and collagen synthesis, complementing the results showing scleraxis expression is induced as fibroblasts (healthy heart) become myofibroblasts (fibrotic heart) (FIG. 6).

To examine whether scleraxis induction is necessary for collagen synthesis in response to stimulation by angiotensin II or TGF-β₁, the inventor will perform RNA interference (RNAi) to block scleraxis production using the BLOCK-iT RNAi adenoviral expression system (Invitrogen). Oligonucleotides for use in RNAi vector preparation will be designed as DNA oligonucleotides encoding small hairpin RNAs (shRNA) in consultation with a commercial advisory service (Invitrogen), then cloned into the pSCREEN-iT vector for assessment of expression knockdown capability (BLOCK-iT RNAi Target Screening System, Invitrogen). This screen works by testing the ability of the shRNA vector to knockdown expression of a scleraxis-β-galactosidase fusion protein in TOP 10 cells, then assaying for P-galactosidase expression. At least three shRNAs will be tested, and the one exhibiting greatest knockdown of expression will be used for all other experiments. The complementary sense strand of this shRNA will be used as a negative control. To generate adenoviral shRNA constructs, the selected shRNA-encoding oligonucleotide will be subcloned into the entry vector pENTR/U6, which will then be recombined with the pAd/BLOCK-iT DEST RNAi Gateway vector as per manufacturer's directions. This vector will then be transfected with Lipofectamine 2000 into 293A packaging-competent cells for amplification and titering of adenovirus.⁵⁴. In a study unrelated to this proposal, the inventor has recently successfully achieved 80% shRNA-mediated knockdown of AKAP84 using the BLOCK-iT system, thus the inventor anticipates no difficulties in using this system for scleraxis (FIG. 10). The scleraxis shRNA adenovirus (AdshScx) will be used in infection experiments similar to those described above: P2 myofibroblasts or P0 fibroblasts will be infected with AdshScx, then 24 hours later will be stimulated with 10⁻⁷ M angiotensin II, or 10 ng/ml TGF-β₁, or vehicle. Collagen production will be assessed 24, 48 or 72 hours after stimulation using picrosirius red, western blotting with SP1.D8 antibodies or PINP determination as described above. The inventor will assess multiple MOIs to ensure the inventor observes the most efficient knockdown of scleraxis expression, as assessed by western blotting with anti-scleraxis antibody or by RT-PCR, as above. Controls will be uninfected, or infected with Ad-LacZ or a scrambled shRNA. The inventor expects that infection with AdshScx will block collagen synthesis in infected myofibroblasts, demonstrating a requirement for scleraxis in collagen synthesis in response to angiotensin II or TGF-β₁.

Experiment 3: Scleraxis Expression Induced in an Animal Model of Cardiac Fibrosis in vivo.

The preliminary in vitro results indicate that phenotypic conversion of fibroblasts to myofibroblasts is associated with a 2-fold increase in scleraxis expression (FIG. 6). The inventor will examine whether scleraxis expression is up-regulated in the intact heart in vivo following MI using a rat model of MI. After sedation of the animals with isoflurane, the chest cavity is opened and the left anterior descending coronary artery is ligated with Cardiopoint 6/0 sutures 2 to 3 mm from the origin, rendering the region distal to the blockage ischemic and resulting in death of the local myocardium¹¹. The chest is then sutured closed and the animals given buprenorphine for analgesia. Within 21 days, infarct scar development is well established, and the scar is mature by 56 days⁵⁸. This model has been well-characterized and closely models human infarct^{14,59,60}. The inventor will perform surgeries on groups of 10 male animals each, since 48 hour mortality is ˜45%, resulting in 5-6 surviving animals per experimental group¹¹; sham animals will be treated as above, but the suture will not be tied. Animals will be monitored by 2D echocardiography (M-mode) to estimate infarct size by performance assessment; only animals with significant (>40%) MI of the left ventricle will be used. Animals will be sacrificed at 1, 7, 21 or 56 days after surgery (total of 40-48 male animals: sham vs. infarct, 1, 7, 21 or 56 days) and the hearts removed. These time points were chosen to assess scleraxis induction immediately after infarct (1 day), in early scar formation (7 days), in established but not yet mature scar (21 days), and in mature scar during the chronic phase of wound healing (56 days). Excised hearts will be trimmed of atria and cut in half along the left anterior descending coronary artery to provide equal amounts of left and right ventricle to each half. Half of the heart will be snap-frozen in OCT freezing medium (Miles) in liquid nitrogen, then serially cryosectioned at 7 μm in a histology core facility. The serial cryosections will be cut to provide a cross-sectional view through the infarct region. The other half will be immediately used to isolate fibroblasts/myofibroblasts as described above. After differential plating to remove cardiomyocytes, the inventor will extract total RNA and perform RT-PCR for scleraxis as described above. Cryosections will be stained with triphenyltetrazolium chloride to visualize the region of infarct⁵¹. Neighbouring sections will be labeled with either anti-scleraxis antibodies (described above) or antibodies to embryonic smooth muscle myosin heavy chain, a marker for myofibroblasts⁶¹. Secondary antibodies used will be conjugated to Texas Red or FITC. Fluorescence will be visualized on a Zeiss M2 epifluorescence microscope in the laboratory. These sections will enable us to examine scleraxis expression in myofibroblasts, and correlate this expression with proximity to the infarcted area in order to examine scleraxis induction in response to MI. Similar sections will be stained for collagen using SP1.D8 antibody to correlate scleraxis expression with collagen synthesis. The inventor has successfully performed immunocytochemistry before (FIG. 9)^{1,62} . As an alternative to using antibodies to scleraxis, in situ hybridization may be performed using a radiolabeled antisense RNA probe for scleraxis as has been described in detail previously². The inventor expects that MI will result in rapid induction of scleraxis throughout the infarct region, and that this elevated expression will remain during scar formation. The inventor also predicts that scleraxis will be abnormally upregulated in areas of the heart distal to the scar, which may contribute to overall cardiac fibrosis.

IV) SUMMARY

The regulation of cardiac ECM formation plays an important role in mediating heart function. This fact is exemplified by the effects of cardiac fibrosis, which dramatically impairs cardiac function and increases patient morbidity and mortality. The mechanisms of many aspects of the initiation and progression of fibrosis have been determined by a host of laboratories working over several decades. Nevertheless, many questions remain about this process, including mechanistic details of how collagen gene expression is regulated at the transcriptional level. The experiments outlined above will examine the potential contribution of the bHLH transcription factor scleraxis to the fibrotic process. Scleraxis has not been previously demonstrated to play a role in the adult myocardium. Furthermore, the role of scleraxis in regulation of collagen gene expression has not been directly examined. The preliminary experiments provide compelling data implicating scleraxis as an important transcriptional regulator of collagen expression in the heart: the inventor has demonstrated that scleraxis is expressed in adult cardiac myofibroblasts; the inventor has shown that scleraxis potently transactivates the promoter of the collagen Iα2 gene, the major cardiac isoform of collagen, likely by binding to E boxes the inventor has identified that are present in this promoter; and the inventor has observed an increase in scleraxis expression in isolated myofibroblasts compared to fibroblasts, and in the adult heart prior to collagen gene induction. The inventor has also developed key tools and techniques for examining the role of scleraxis in cardiac collagen synthesis, including RT-PCR for quantification of scleraxis transcripts, and an adenovirus encoding scleraxis for infection of myofibroblasts.

The inventor proposes a comprehensive series of experiments to study these issues in detail. The experiments described in Experiment 1, together with the preliminary data (FIGS. 4 and 5), will examine whether scleraxis is sufficient to regulate expression of collagen Iα2, one of the main collagens expressed in the fibrotic heart and a primary component of the healthy ECM. The experiments described in Experiment 2 will examine whether scleraxis is necessary for the fibrotic response of myofibroblasts to known pro-fibrotic factors. Finally, the experiments described in Experiment 3 will examine whether scleraxis expression increases during post-MI scar formation, a reasonable expectation if scleraxis is a key regulator of fibrosis. Together, these experiments will provide a comprehensive examination of the role and mechanism of scleraxis in cardiac fibrosis.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE I
Collagen gene expression in acute cardiac failure transgenic mice.
Total cardiac cDNA from induced or non-induced transgenic mice
expressing HDAC5S/A was isolated at 1 or 4 days following transgene
induction, then analyzed on Affymetrix U74Av2 DNA microarrays.
Fold change is reported relative to control non-induced transgenic
mice. Collagens Iα1, Iα2, IIIα1, Vα2 and VIIIα1 are associated
with cardiac fibrosis, while IIα1, IXα2 and XIα1 are not.

Expression Level (fold change)

| Gene | 1 day | 4 days |
|---|---|---|
| Scleraxis | 2.5 | 3.5 |
| Procollagen Iα1 | N.C. | 2.3 |
| Procollagen Iα2 | N.C. | 2.3 |
| Procollagen IIIα1 | N.C. | 2.0 |
| Procollagen Vα2 | N.C. | 2.5 |
| Collagen VIIIα1 | N.C. | 3.5 |
| Collagen IIα1 | N.C. | N.C. |
| Procollagen IXα2 | N.C. | N.C. |
| Procollagen XIα1 | N.C. | N.C. |

TABLE II

Putative E-boxes within the
collagen Iα2 promoter.

| | | | |
|---|---|---|---|
| Position | Sequence | Position | Sequence |

| | | | |
|---|---|---|---|
| −3317 | CATGTG | −1638 | CACTTG |
| −2866 | CAGCTG | −847 | CAGGTG |
| −2768 | CAAATG | −762 | CACCTG |
| −2708 | CATTTG | −494 | CAAGTG |
| −2282 | CATATG | −476 | CAGGTG |
| −1888 | CAAGTG | −178 | CAGCTG |

Italicized sequences have previously been demonstrated to bind scleraxis with high affinity. Positions are relative to the 3′ end of the promoter sequence, which is at −80 relative to the ATG start codon of collagen Iα2.

TABLE III
Characteristics of Fibroblasts/Myofibroblasts at Different Passage
Numbers. Primary isolated cardiac fibroblasts (PO) secrete some
collagen, but are non-contractile and do not express α-SMA. As
passage number increases, cells undergo phenotypic conversion,
first to intermediary promyofibroblasts (P1) and then to
myofibroblasts (P2), which express α-SMA and SM_{emb},
are highly contractile, synthesize stress fibres and secrete
significant amounts of collagen⁹.

| | P0 | P1 | P2 |
|---|---|---|---|
| Contractility | − | + | ++ |
| Expression of SM_{emb} | − | + | ++ |
| Expression of α-smooth muscle actin | − | + | ++ |
| Collagen synthesis | + | ++ | +++ |
| Stress fibre formation | − | + | ++ |

### Full Citations for References Referred to in the Specification

1. Czubryt, M. P., McAnally, J., Fishman, G. I. & Olson, E. N. Regulation of peroxisome proliferator-activated receptor gamma coactivator 1 alpha (PGC-1 alpha) and mitochondrial function by MEF2 and HDAC5. Proc. Natl. Acad. Sci. U. S. A. 100, 1711-6 (2003).
2. Cserjesi, P. et al. Scleraxis: a basic helix-loop-helix protein that prefigures skeletal formation during mouse embryogenesis. Development 121, 1099-110 (1995).
3. Lincoln, J., Alfieri, C. M. & Yutzey, K. E. Development of heart valve leaflets and supporting apparatus in chicken and mouse embryos. Dev Dyn 230, 239-50 (2004).
4. Liu, Y. et al. Overexpression of a single helix-loop-helix-type transcription factor, scleraxis, enhances aggrecan gene expression in osteoblastic osteosarcoma ROS17/2.8 cells. J Biol Chem 272, 29880-5 (1997).
5. Swynghedauw, B. Molecular mechanisms of myocardial remodeling. Physiol Rev 79, 215-62 (1999).
6. Colucci, W. S. & Braunwald, E. Pathophysiology of heart failure. in Heart Disease (ed. Braunwald, E.) 394-420 (W.B. Saunders Company, Philadelphia, 1997).
7. Kannel, W. B. Epidemiology of heart failure in the United States. in Heart Failure (eds. Poole-Wilson, P. A., Colucci, W. S., Massie, B. M., Chatterjee, K. & Coats, A. J. S.) 279-288 (Churchill Livingstone, N.Y., 1997).
8. Anversa, P., Beghi, C., Kikkawa, Y. & Olivetti, G. Myocardial infarction in rats.

Infarct size, myocyte hypertrophy, and capillary growth. Circ Res 58, 26-37 (1986).
9. Freed, D. H., Cunnington, R. H., Dangerfield, A. L., Sutton, J. S. & Dixon, I. M.

Emerging evidence for the role of cardiotrophin-1 in cardiac repair in the infarcted heart. Cardiovasc Res 65, 782-92 (2005).
10. Cleutjens, J. P., Verluyten, M. J., Smiths, J. F. & Daemen, M. J. Collagen remodeling after myocardial infarction in the rat heart. Am J Pathol 147, 325-38 (1995).
11. Ju, H., Zhao, S., Tappia, P. S., Panagia, V. & Dixon, I. M. Expression of Gq alpha and PLC-beta in scar and border tissue in heart failure due to myocardial infarction. Circulation 97, 892-9 (1998).
12. Pfeffer, M. A. & Braunwald, E. Ventricular remodeling after myocardial infarction. Experimental observations and clinical implications. Circulation 81, 1161-72 (1990).
13. Pfeffer, M. A., Pfeffer, J. M., Steinberg, C. & Finn, P. Survival after an experimental myocardial infarction: beneficial effects of long-term therapy with captopril. Circulation 72, 406-12 (1985).
14. Feild, B. J. et al. Left ventricular size and function and heart size in the year following myocardial infarction. Circulation 50, 331-9 (1974).
15. Messier, R. H., Jr. et al. Dual structural and functional phenotypes of the porcine aortic valve interstitial population: characteristics of the leaflet myofibroblast. J Surg Res 57, 1-21 (1994).
16. Weber, K. T. & Brilla, C. G. Pathological hypertrophy and cardiac interstitium.

Fibrosis and renin-angiotensin-aldosterone system. Circulation 83, 1849-65 (1991).
17. Powell, D. W. et al. Myofibroblasts. I. Paracrine cells important in health and disease. Am. J Physiol. 277, C1-9 (1999).
18. Eghbali, M. et al. Collagen chain mRNAs in isolated heart cells from young and adult rats. J Mol Cell Cardiol 20, 267-76 (1988).
19. Willems, I. E., Havenith, M. G., De Mey, J. G. & Daemen, M. J. The alpha-smooth muscle actin-positive cells in healing human myocardial scars. Am J Pathol 145, 868-75 (1994).
20. Hao, J., Wang, B., Jones, S. C., Jassal, D. S. & Dixon, I. M. Interaction between angiotensin II and Smad proteins in fibroblasts in failing heart and in vitro. Am J Physiol Heart Circ Physiol 279, H3020-30 (2000).
21. Wang, B. et al. Decreased Smad 7 expression contributes to cardiac fibrosis in the infarcted rat heart. Am J Physiol Heart Circ Physiol 282, H1685-96 (2002).
22. Weber, K. T. Fibrosis, a common pathway to organ failure: angiotensin II and tissue repair. Semin Nephrol 17, 467-91 (1997).
23. Sun, Y. & Weber, K. T. Angiotensin II receptor binding following myocardial infarction in the rat. Cardiovasc Res 28, 1623-8 (1994).
24. Hildebrand, A. et al. Interaction of the small interstitial proteoglycans biglycan, decorin and fibromodulin with transforming growth factor beta. Biochem J 302 (Pt 2), 527-34 (1994).
25. Brand, T. & Schneider, M. D. Transforming growth factor-beta signal transduction. Circ Res 78, 173-9 (1996).
26. Brand, T. & Schneider, M. D. The TGF beta superfamily in myocardium: ligands, receptors, transduction, and function. J Mol Cell Cardiol 27, 5-18 (1995).
27. Kingsley, D. M. The TGF-beta superfamily: new members, new receptors, and new genetic tests of function in different organisms. Genes Dev 8, 133-46 (1994).
28. Inagaki, Y., Truter, S. & Ramirez, F. Transforming growth factor-beta stimulates alpha 2(I) collagen gene expression through a cis-acting element that contains an Spl-binding site. J Biol Chem 269, 14828-34 (1994).
29. Sadoshima, J. & Izumo, S. Molecular characterization of angiotensin II—induced hypertrophy of cardiac myocytes and hyperplasia of cardiac fibroblasts. Critical role of the AT1 receptor subtype. Circ Res 73, 413-23 (1993).
30. Ohta, K. et al. Role of angiotensin II in extracellular matrix and transforming growth factor-beta 1 expression in hypertensive rats. Eur J Pharmacol 269, 115-9 (1994).
31. Dixon, I. M., Hao, J., Reid, N. L. & Roth, J. C. Effect of chronic AT(1) receptor blockade on cardiac Smad overexpression in hereditary cardiomyopathic hamsters. Cardiovasc Res 46, 286-97 (2000).
32. Brooks, W. W. & Conrad, C. H. Myocardial fibrosis in transforming growth factor beta(1)heterozygous mice. J Mol Cell Cardiol 32, 187-95 (2000).
33. Schiller, M., Javelaud, D. & Mauviel, A. TGF-beta-induced SMAD signaling and gene regulation: consequences for extracellular matrix remodeling and wound healing. J Dermatol Sci 35, 83-92 (2004).
34. Molkentin, J. D. & Olson, E. N. Combinatorial control of muscle development by basic helix-loop-helix and MADS-box transcription factors. Proc Natl Acad Sci USA 93, 9366-73 (1996).
35. Carlberg, A. L., Tuan, R. S. & Hall, D. J. Regulation of scleraxis function by interaction with the bHLH protein E47. Mol Cell Biol Res Commun 3, 82-6 (2000).
36. Brown, D., Wagner, D., Li, X., Richardson, J. A. & Olson, E. N. Dual role of the basic helix-loop-helix transcription factor scleraxis in mesoderm formation and chondrogenesis during mouse embryogenesis. Development 126, 4317-29 (1999).
37. Schweitzer, R. et al. Analysis of the tendon cell fate using Scleraxis, a specific marker for tendons and ligaments. Development 128, 3855-66 (2001).
38. Brent, A. E., Schweitzer, R. & Tabin, C. J. A somitic compartment of tendon progenitors. Cell 113, 235-48 (2003).
39. Dubrulle, J. & Pourquie, 0. Welcome to syndetome: a new somitic compartment. Dev Cell 4, 611-2 (2003).
40. Brent, A. E., Braun, T. & Tabin, C. J. Genetic analysis of interactions between the somitic muscle, cartilage and tendon cell lineages during mouse development. Development 132, 515-28 (2005).
41. Asou, Y. et al. Coordinated expression of scleraxis and Sox9 genes during embryonic development of tendons and cartilage. J Orthop Res 20, 827-33 (2002).
42. Muir, T., Sadler-Riggleman, I. & Skinner, M. K. Role of the basic helix-loop-helix transcription factor, scleraxis, in the regulation of Sertoli cell function and differentiation. Mol Endocrinol 19, 2164-74 (2005).
43. Watanabe, H. et al. Mouse cartilage matrix deficiency (cmd) caused by a 7 bp deletion in the aggrecan gene. Nat Genet 7, 154-7 (1994).
44. Zur Nieden, N. I., Kempka, G., Rancourt, D. E. & Ahr, H. J. Induction of chondro-, osteo- and adipogenesis in embryonic stem cells by bone morphogenetic protein-2: Effect of cofactors on differentiating lineages. BMC Dev Biol 5, 1 (2005).
45. Salingcarnboriboon, R. et al. Establishment of tendon-derived cell lines exhibiting pluripotent mesenchymal stem cell-like property. Exp Cell Res 287, 289-300 (2003).
46. Mendler, M., Eich-Bender, S. G., Vaughan, L., Winterhalter, K. H. & Bruckner, P. Cartilage contains mixed fibrils of collagen types II, IX, and XI. J Cell Biol 108, 191-7 (1989).
47. Liu, Y., Cserjesi, P., Nifuji, A., Olson, E. N. & Noda, M. Sclerotome-related helix-loop-helix type transcription factor (scleraxis) mRNA is expressed in osteoblasts and its level is enhanced by type-beta transforming growth factor. J Endocrinol 151, 491-9 (1996).
48. lavarone, A., Garg, P., Lasorella, A., Hsu, J. & Israel, M. A. The helix-loop-helix protein Id-2 enhances cell proliferation and binds to the retinoblastoma protein. Genes Dev 8, 1270-84 (1994).
49. Sun, X. H., Copeland, N. G., Jenkins, N. A. & Baltimore, D. Id proteins Id1 and Id2 selectively inhibit DNA binding by one class of helix-loop-helix proteins. Mol Cell Biol 11, 5603-11 (1991).
50. Freed, D. H., Borowiec, A. M., Angelovska, T. & Dixon, I. M. Induction of protein synthesis in cardiac fibroblasts by cardiotrophin-1: integration of multiple signaling pathways. Cardiovasc Res 60, 365-75 (2003).
51. Peterson, D. J. et al. Expression of Gi-2 alpha and Gs alpha in myofibroblasts localized to the infarct scar in heart failure due to myocardial infarction. Cardiovasc Res 41, 575-85 (1999).
52. Sun, M. et al. Excessive tumor necrosis factor activation after infarction contributes to susceptibility of myocardial rupture and left ventricular dysfunction. Circulation 110, 3221-8 (2004).
53. Perez, A. V., Perrine, M., Brainard, N. & Vogel, K. G. Scleraxis (Scx) directs lacZ expression in tendon of transgenic mice. Mech Dev 120, 1153-63 (2003).
54. Hurtado, C. et al. Adenovirally delivered shRNA strongly inhibits Na+—Ca2+ exchanger expression but does not prevent contraction of neonatal cardiomyocytes. J Mol Cell Cardiol 38, 647-54 (2005).
55. Regula, K. M., Ens, K. & Kirshenbaum, L. A. IKK beta is required for Bcl-2-mediated NF-kappa B activation in ventricular myocytes. J Biol Chem 277, 38676-82 (2002).
56. Regula, K. M. & Kirshenbaum, L. A. p53 activates the mitochondrial death pathway and apoptosis of ventricular myocytes independent of de novo gene transcription. J Mol Cell Cardiol 33, 1435-45 (2001).
57. Kirshenbaum, L. A. Bcl-2 intersects the NFkappaB signalling pathway and suppresses apoptosis in ventricular myocytes. Clin Invest Med 23, 322-30 (2000).
58. Robinson, T. F., Factor, S. M. & Sonnenblick, E. H. The heart as a suction pump. Sci Am 254, 84-91 (1986).
59. Fletcher, P. J., Pfeffer, J. M., Pfeffer, M. A. & Braunwald, E. Left ventricular diastolic pressure-volume relations in rats with healed myocardial infarction. Effects on systolic function. Circ Res 49, 618-26 (1981).
60. Fletcher, P. J., Pfeffer, J. M., Pfeffer, M. A. & Braunwald, E. Effects of hypertension on cardiac performance in rats with myocardial infarction. Am J Cardiol 50, 488-96 (1982).
61. Frangogiannis, N. G., Michael, L. H. & Entman, M. L. Myofibroblasts in reperfused myocardial infarcts express the embryonic form of smooth muscle myosin heavy chain (SMemb). Cardiovasc Res 48, 89-100 (2000).
62. Czubryt, M. P., Austria, J. A. & Pierce, G. N. Hydrogen peroxide inhibition of nuclear protein import is mediated by the mitogen-activated protein kinase, ERK2. J Cell Biol 148, 7-15 (2000).

I claim:

1. A method for modulating collagen synthesis comprising administering an effective amount of a scleraxis modulator to a cell or animal in need thereof.

2. The method according to claim 1 comprising administering an effective amount of scleraxis antagonist to inhibit collagen synthesis.

3. The method according to claim 2 wherein the antagonist is selected from the group consisting of an antibody fragment, small molecule, peptide mimetic, peptide or an antisense oligonucleotide to scleraxis.

4. The method according to claim 2 wherein the antagonist interferes with the binding of scleraxis to a promoter region of collagen Iα2.

5. The method according to claim 4 wherein the antagonist interferes with binding of scleraxis to an E-box region of the promoter region of collagen Iα2.

6. The method according to claim 2 wherein the antagonist: interferes with the binding of scleraxis to DNA encoding scleraxis target genes, inhibits scleraxis transactivational activity, and/or inhibits scleraxis interaction with other proteins, optionally co-regulator proteins or binding partner proteins.

7. The method according to claim 4 wherein the antagonist inhibits the binding of scleraxis to: the sequence CATGTG found at nucleotide position −3317; the sequence CAGGTG found at nucleotide position −847; or the sequence CAGGTG found a nucleotide position −476, relative to the 3′ of the promoter sequence which is at −80 relative to the ATG start codon of collagen Iα2.

8. The method according to claim 2 for the treatment or prevention of fibrosis.

9. The method according to claim 3 for the treatment or prevention of fibrosis.

10. A method according to claim 8 for the treatment or prevention of cardiac fibrosis.

11. A method according to claim 9 for the treatment or prevention of cardiac fibrosis.

12. The method according to claim 1 comprising administering an effective amount of scleraxis agonist to stimulate collagen synthesis.

13. The method according to claim 12 to assist in wound healing, improve skin grafts and to combat the effects of aging.

14. A method for identifying a compound that modulates collagen synthesis comprising:
(a) incubating a test compound with a cell expressing scleraxis; and
(b) determining the effect of the compound on scleraxis activity or expression and comparing with a control, wherein a change in scleraxis activity or expression as compared to the control indicates that the test compound may modulate collagen synthesis.

15. The method according to claim 14 for identifying an antagonist of scleraxis useful in inhibiting collagen synthesis comprising the steps of:
(a) incubating a test compound with scleraxis; and
(b) determining whether or not the test compound inhibits scleraxis, wherein inhibition of the scleraxis indicates that the compound is a scleraxis antagonist and may be useful in inhibiting collagen synthesis.
